(19) United States
(12) Patent Application Publication
(10) Pub. No.: US 2009/0084801 A1
(43) Pub. Date: Apr. 2, 2009

(54) CHILD-RESISTANT COMPLIANCE PILL BOTTLE

(75) Inventor: Matthew Coe, Annandale, NJ (US)

Correspondence Address: DUANE MORRIS LLP - Philadelphia;IP DEPARTMENT, 30 SOUTH 17TH STREET, PHILADELPHIA, PA, 19103-4196, US

(73) Assignee: One World Design & Manufacturing Group Ltd., Warren, NJ (US)

(21) Appl. No.: 12/235,223

(22) Filed: Sep. 22, 2008

Related U.S. Application Data
(60) Provisional application No. 60/975,658, filed on Sep. 27, 2007.
(60) Provisional application No. 60/980,818, filed on Oct. 18, 2007.

(51) Int. Cl. A61B 19/02; B65D 55/02

(52) U.S. Cl. 220/740; 215/223

(57) ABSTRACT

A pill bottle for housing a blister card that includes a top section, a bottom section, and a center section connected by a pair of hinges. The top section and bottom section each include one half of a child-resistant neck. One half of the neck including a member that can be rotated while being connected to a knob having a hook on a bottom surface. The other half of the neck has two stacked platforms with one of the platforms having a slot and an opening. The hook lines up with and enters the opening to lock the pill bottle closed unless the knob is pressed so the hook drops too low for the opening and then rotated so the hook can slide out of the slot. This allows the top and bottom sections to open and reveal a blister card attached to the center section of the pill bottle.

This application claims priority from, and the benefit of U.S. provisional patent application Ser. No. 60/975,658, filed Sep. 27, 2007, entitled Compliance Pill Bottle and U.S. provisional patent application, and U.S. provisional patent application Ser. No. 60/980,818, filed Oct. 18, 2007, and entitled Child Resistant Compliance Pill Bottle, all of which are incorporated herein by reference.

### FIELD OF INVENTION

The present invention generally relates to packaging suitable for storing and dispensing medication; and more specifically to medication bottles that provide a simple and familiar way for a user to take medication as well as determine if a dose has been taken while being senior friendly and meeting child resistance standards.

### BACKGROUND OF THE INVENTION

It is well known that medications, such as pills, tablets, and capsules, must be administered over a dosing period. In many circumstances, the medications can be harmful to children, so law-makers have passed regulations concerning child resistant packaging. The use of child resistant packaging is well known throughout the art, and has been utilized for goods ranging from household items to pharmaceutical products. The pharmaceutical industry has settled on two main methods of dispensing medication, either loosely in a “amber” bottle or more discretely in a blister card.

The traditional child-resistant “amber” bottle utilizes a “push and turn” cap by which the person wishing to open the bottle must press down to release a locking mechanism before turning the cap. These bottles are effective for mass distribution of a medication, but have significant drawbacks. First, “push and turn” cap's are often smaller in size, so that they do not provide a sufficiently stable gripping surface to allow a user of limited dexterity, i.e. the elderly, to access the medication in side easily. Secondly, the level of chemical stability of the individual pills has been a serious concern. Pills that are dispensed in traditional “amber bottles” are often handled and put back into the bottle or, since the bottle is being constantly opened and closed, excessive contamination and moisture often enter the bottle. For example, hand moisture and body oils can significantly reduce the shelf-life and effectiveness of a medication. Finally, “amber bottles” provide no compliance feature that informs the user as to whether or not a dose has been taken during that dosing period, be it a morning, afternoon, day, or week.

The other well-known medication packaging is “unit dose” packaging or blister cards. Blister cards are typically formed from flexible materials with a plurality of cavities that receive and dispense one pill. An open side of each cavity is covered with a pierceable foil seal. The user simply pushes a selected pill through the foil seal in order to remove the medication from its individual blister on the card. Blister card packaging ensures stability and allows for patient compliance, although it often does not provide sufficient protection for the medication or child resistance.

### SUMMARY OF THE INVENTION

The present invention provides a senior-friendly, child-resistant pill bottle that includes a blister card and a rotational “push and turn” cap. In one general aspect, a pill bottle includes a front panel and a back panel that connect to a central panel. Each panel includes a neck portion that together align to form the inventive child resistant feature. In one embodiment, the neck portions align to allow a separable cap or knob to be placed over and around the neck portions so as to snugly fit receive them. The neck portions align to form a child resistant “push and turn” cap receiving area. A child resistant cap is then placed upon the combined neck portions thereby sealing a blister card inside the bottle. The bottle, when opened, separates into two nearly identical halves. Each half is arranged to receive at least one blister card. The at least one blister card is attached to the container permanently.

In another embodiment, the child resistant “push and turn” cap is an integral feature of a bottle that provides for a one piece design. In this embodiment, one of the neck portions includes a slot and aperture, while another neck portion is outfitted with a knob that is connected to a flexible stand that is capable of being both pressed and rotated. The knob or cap includes a hook that projects from the bottom surface of the knob. When closing the pill bottle, the user will press and rotate the knob so that the hook aligns with the slot in the neck portion. Upon releasing the knob, it will re-align itself with the rest of the bottle and so that the hook is aligned with the aperture in the neck and latch. This construction acts to seal a blister card or cards within the pill bottle. To open, the user presses down and turns the knob thereby releasing the hook from the aperture and aligning it with the slot in the neck so as to release the child resistant feature.

### BRIEF DESCRIPTION OF THE DRAWINGS

These and other features and advantages of the present invention will be more fully disclosed in, or rendered obvious by the following detailed description of preferred embodiments of the invention, which are to be considered together with the accompanying drawings wherein like numbers refer to like parts, and further wherein:

FIG. 1 is a front perspective view of an child resistant pill bottle with attached blister card in accordance with the present invention;

FIG. 2 is a perspective front view of the child resistant pill bottle shown in FIG. 1;

FIG. 3 is a front perspective view of the child resistant pill bottle shown in FIG. 1, in a partially open position;

FIG. 4 is a perspective from view of the child resistant pill bottle shown in FIG. 1, in an open position;

FIG. 5 is a top view of the child resistant pill bottle shown in FIG. 4;

FIG. 6 is a side view of the child resistant pill bottle shown in FIG. 4;

FIG. 7 is a cross-sectional view of an open child resistant pill bottle shown in FIG. 4, as taken along lines 100-100 in FIG. 5;

FIG. 8 is a back view of the open child resistant pill bottle shown in FIG. 1;

FIG. 9. is a side view of an open child resistant pill bottle shown in FIG. 8;

FIG. 10 is a cross-sectional view of the child resistant pill bottle shown in FIG. 8, as taken along lines 200-200 in FIG. 8;

FIG. 11 is a front top perspective view of an open child resistant pill bottle with a blister card attached in accordance with the present invention;

FIG. 12 is a front perspective view of the child resistant pill bottle in accordance with the present invention;

FIG. 13 is a front view of the child resistant pill bottle shown in FIG. 12;

FIG. 14 is a side view of the closed child resistant pill bottle shown in FIG. 12;

FIG. 15 is a back view of an open child resistant pill bottle as shown in FIG. 12;

FIG. 16 is a front perspective view of an open child resistant pill bottle with blister card shown in FIG. 12; and

FIG. 17 is an exploded side view of the child resistant pill bottle shown in FIG. 16.

### DETAILED DESCRIPTION OF THE PREFERRED EMBODIMENTS

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

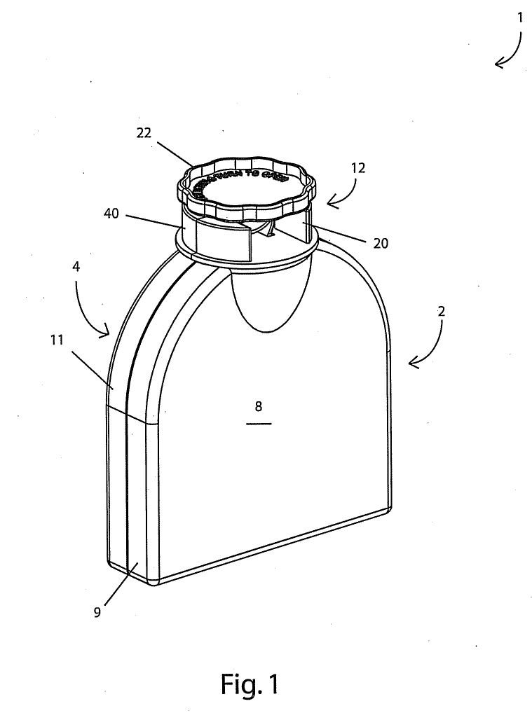

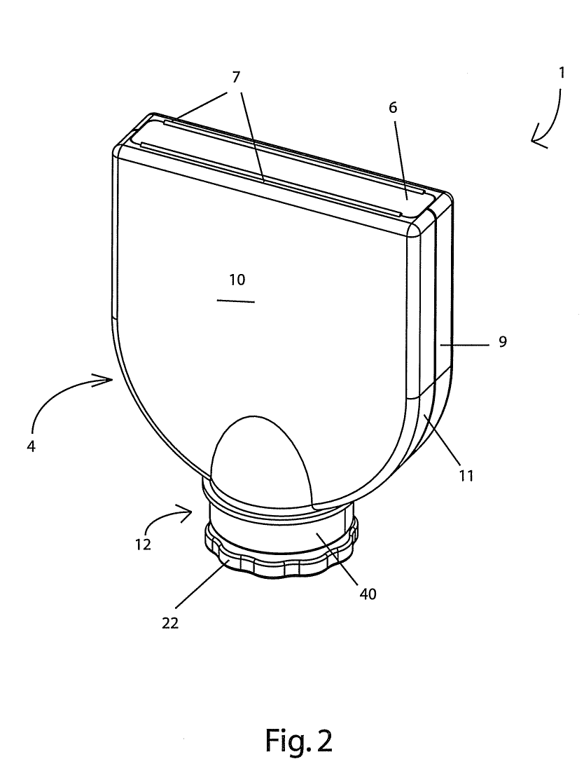

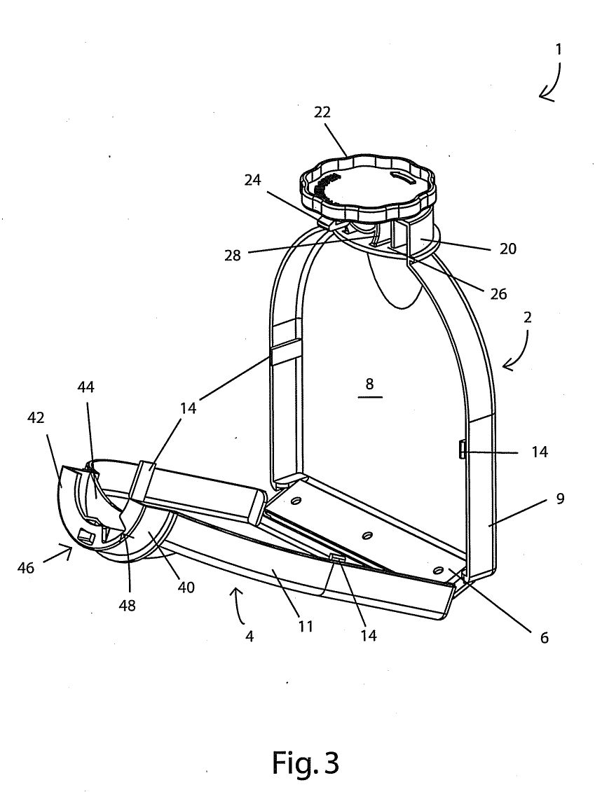

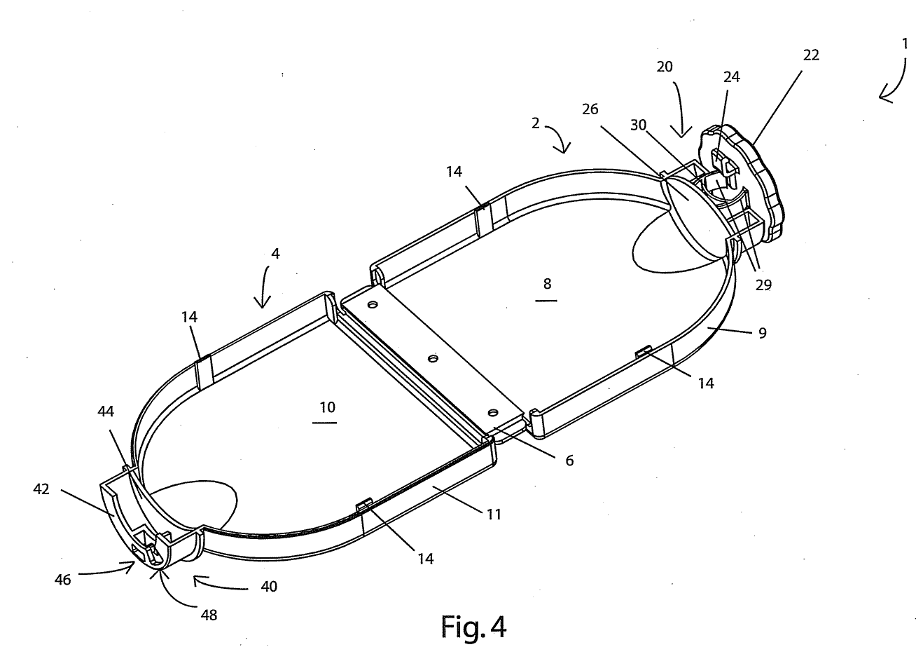

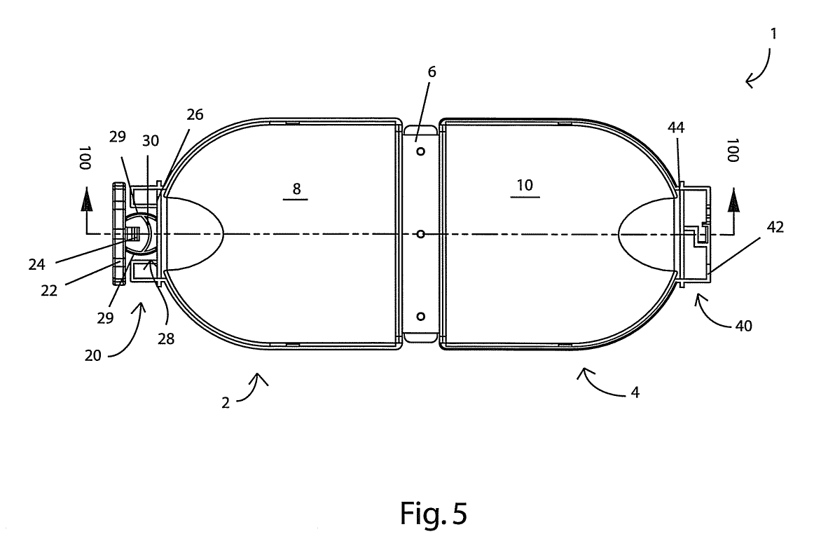

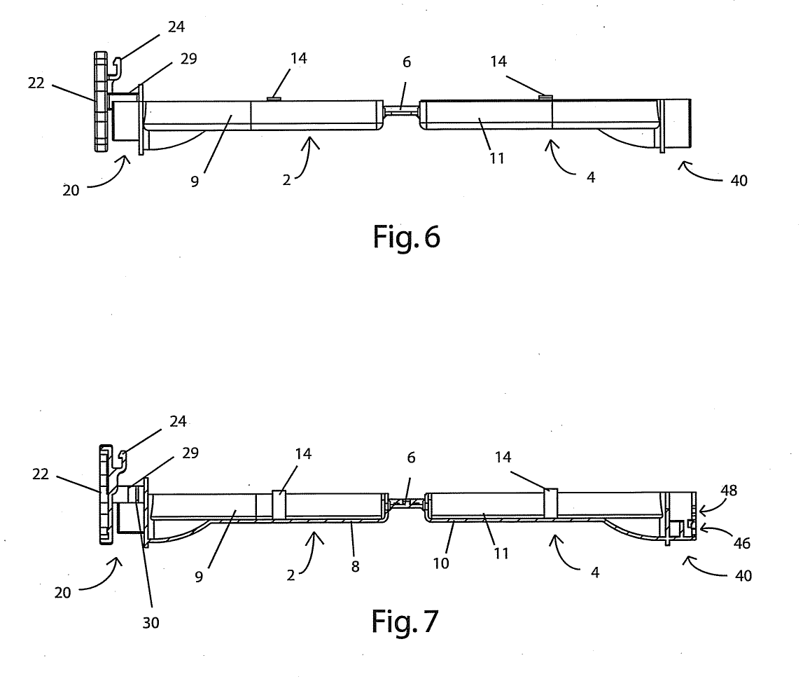

Referring to FIGS. 1-7, a senior-friendly, child-resistant pill bottle 1 includes a top section 2, a bottom section 4, center section 6 and a child-resistant neck 12. Top section 2 and bottom section 4 are movably fastened to center section 6 by two, spaced apart living hinges 7. Top section 2 includes a top panel 8 and top sidewalls 9 that taper at an end where they join together to form a portion of child-resistant neck 12. Similarly, bottom section 4 includes bottom panel 10 and curved sidewalls 11 that taper at an end where they join together to form another portion of child-resistant neck 12. Thus, child-resistant neck 12 is formed by the joining of the two tapered portions of top section 2 and bottom section 4 to form top neck 20 and bottom neck 40. Alignment snaps 14 are molded into top sidewall 9 and bottom sidewall 11 so as to allow for a releasable closure.

Top neck 20 is formed from the two outer walls that surround an alignment platform 26, which rests atop top sidewalls 9 thereby forming the base of top neck 20, a rotator 28, a knob 22, and a hook 24. Rotator 28 is connected to the top of alignment platform 28 by two curved legs 29. A curved cross-member 30 connects curved legs 29. Knob 22 is connected to opposite ends of curved outer legs 29. The alignment of curved legs 29 and curved cross member 30 allow knob 22 to be both flexed downwardly, via pressure applied to the top of the knob 22 by the user, and turned about alignment platform 28. Hook 24 is connected to or integrally formed with a bottom surface of knob 22. Hook 24 is constructed so as to bend forwardly and upwardly, while being integral with knob 22 while also rotating and flexing along with knob 22. Bottom neck 40 includes an outer wall that circumferentially surrounds a recess platform 44, which rests atop back sidewalls 11 thereby forming a base for bottom neck 40. An upper platform 42 is connected to recess platform 44 via circumferential back neck wall 40. Slot 48 and hook opening 46 are both formed in upper platform 42.

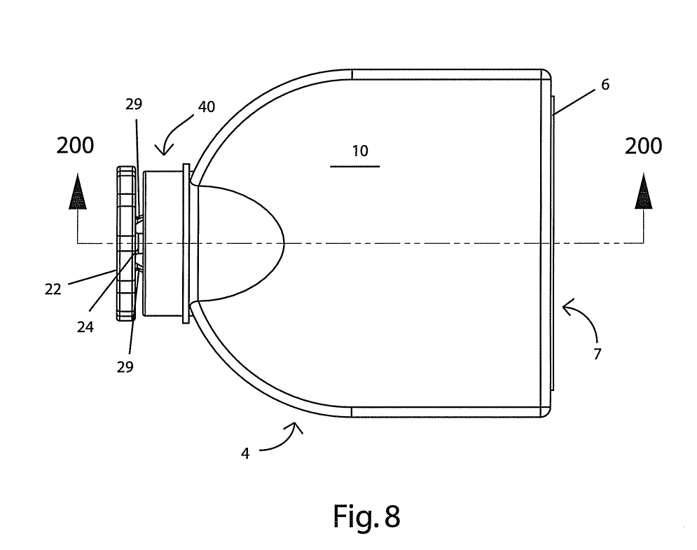

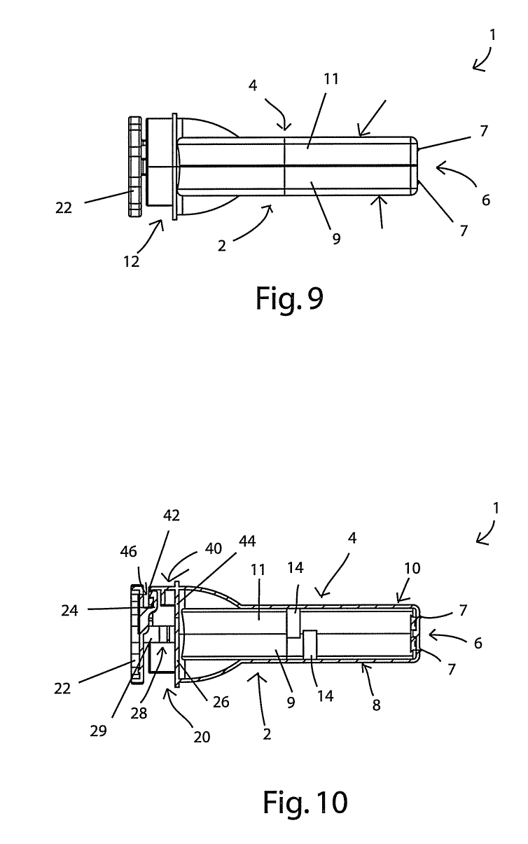

Referring to FIGS. 8-10, when pill bottle 1 is in a closed position, the edges of alignment platform 26 and recess platform 44 make contact, thereby causing top neck 20 and bottom neck 40 to align and form child-resistant neck 12. Alignment snaps 14 are oriented so as to fit over a slight lip formed in either bottom sidewall 11 or top sidewall 9, respectively. Hook 22 locks within hook opening 46, thereby preventing top section 2 and bottom section 4 from separating. Pressing down upon knob 22 thereby flexing curved legs 29 downwardly until hook 24 is below hook opening 46, opens pill bottle 1. Knob 22 is then rotated to the left, engaging rotator 28 until hook 24 aligns with slot 48. The top section 2 and bottom section 4 may then be separated to gain access to pill bottle 1.

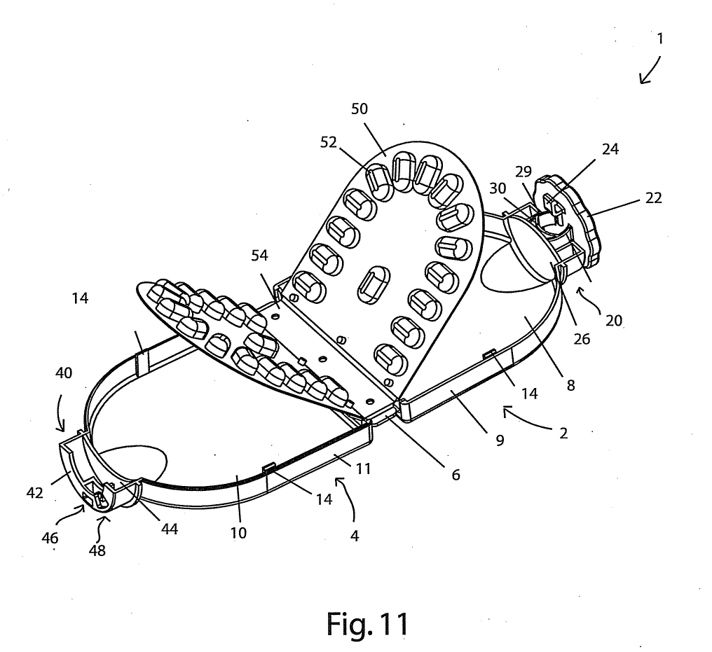

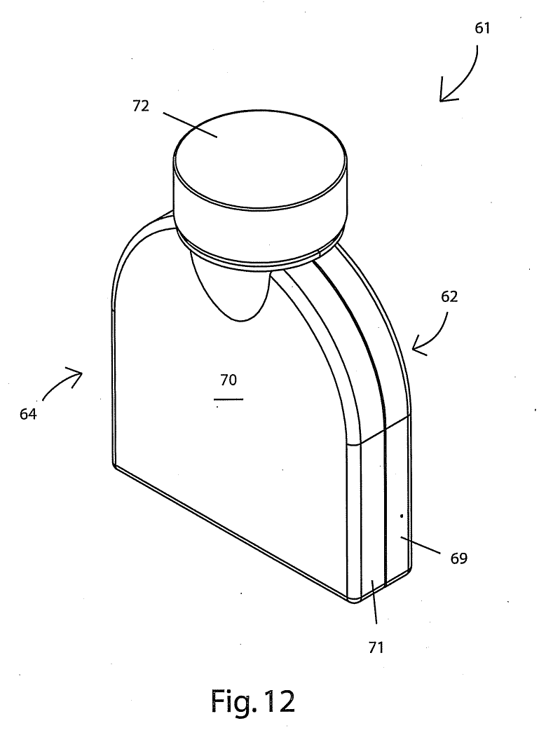

Referring to FIG. 11, blister card 50, charged with medication 52 includes a blister center 54 that interconnects with center section 6. Blister card 50 is hinged so as to bend either independently of or in conjunction with the top section 2 or bottom section 4. Blister center 54 of blister card 50 is often connected to center section 6 via any appropriate means including but not limited to heat staking.

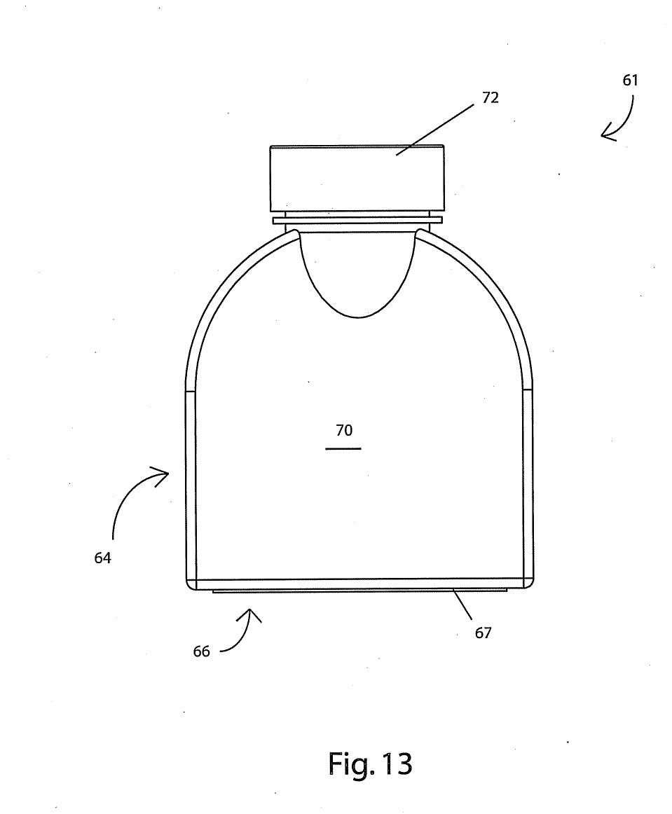

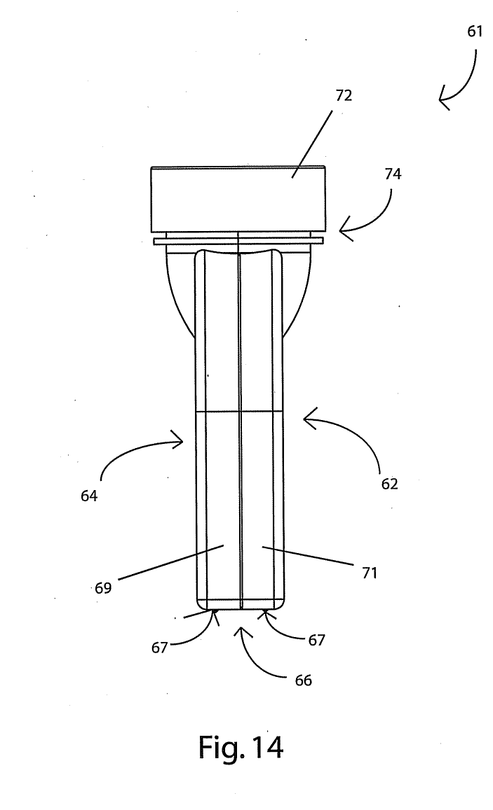

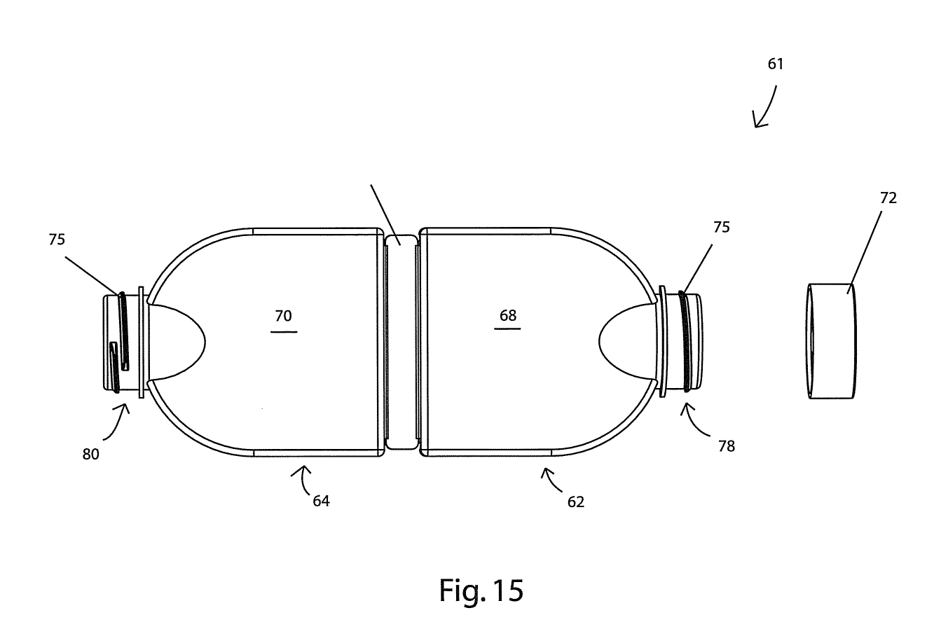

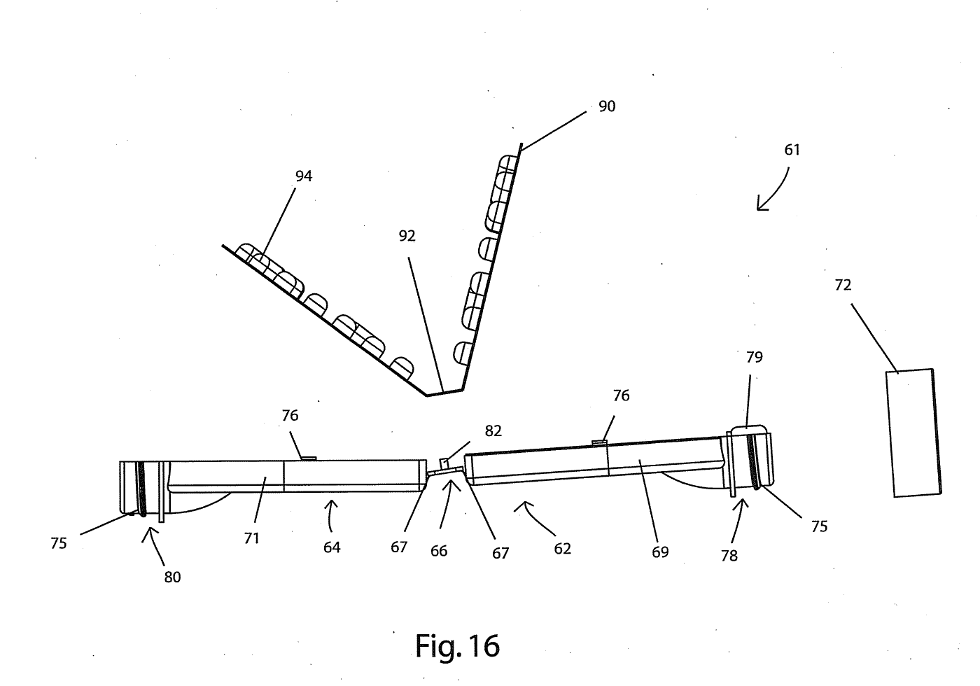

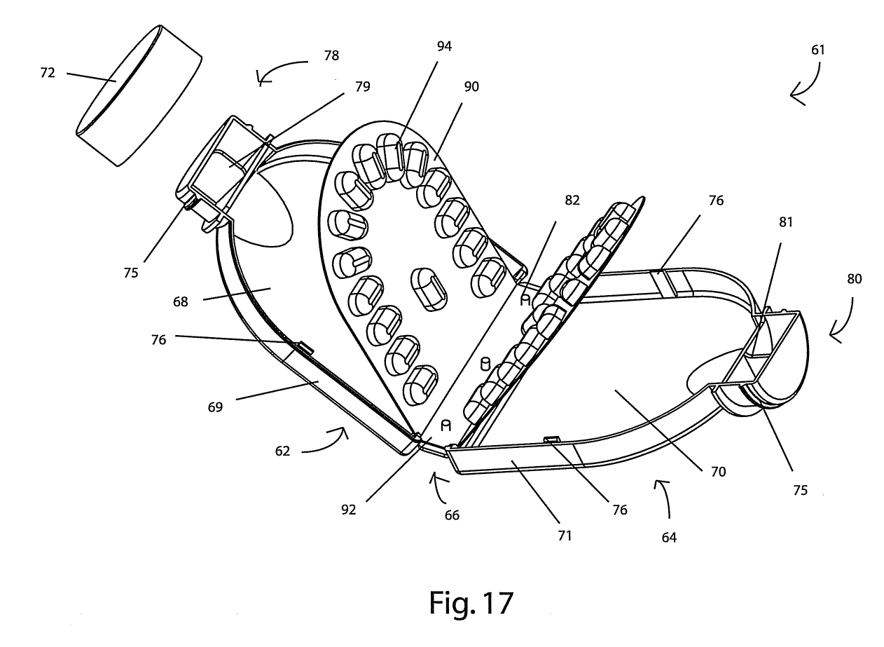

Referring to FIGS. 12-14, an alternative senior-friendly, child-resistant pill bottle 61 includes a top section 62 and a bottom section 64 that are connected to center section 66 by hinges 67. Top section 62 includes top panel 68 and top sidewalls 69, and bottom section 64 includes bottom panel 70 and bottom sidewalls 71. Top section 62 and bottom section 64 are molded to form child-resistant neck 74 having thread 75. Cap 72 rests upon child-resistant neck 74 and holds top section 62 and bottom section 64 abutting one another and pill bottle 61 closed. Referring to FIGS. 15-17, yet another embodiment of senior-friendly, child-resistant pill bottle 61 includes a top section 62 and a bottom section 64, each having top neck 78 and bottom neck 80 respectively, comprising a one half of thread 75 molded in an exterior circular surface. Cap 72 has a similar internal half thread (not shown) providing child-resistant cap. Alignment tab 79 is arranged in perpendicular relation to inside top neck 78, and coincides with recess tab 81, which rests in a similar position within bottom neck 80. Alignment tab 79 extends beyond the inner surface of top neck 78 and enters a space left within back neck 80 by an inward curve in recess tab 81. The alignment tab 79 and recess tab 81 cooperate to provide a stable surface for cap 72 to be removed and ensure stability of the pill bottle 61, when in a closed position. (FIGS. 12-14) Alignment clips 74 are molded in top sidewalls 69 and bottom sidewalls 71 and snap onto their opposite sidewall to improve the structural integrity of pill bottle 61. Posts 82 are arranged perpendicular to the inner surface of center section 66.

In a further embodiment, blister card 90 includes a blister center 92 and a blister opening 96 that aligns with posts 82. Blister card 90 is then attached to the pill bottle by fitting blister openings 96 in blister center 92 and over posts 82 in center section 66. Posts 82 may turn be heat staked.

It is to be understood that the present invention is by no means limited to the particular constructions herein disclosed and shown in the drawings, but also comprises any modifications or equivalents within the scope of the claims.

What is claimed is:

1. A pill bottle for housing a blister card comprising:
a top section including a front panel;
a bottom section including a back panel;
a center section connected to said front panel and back panel by hinges;
a child-resistant neck including:
an alignment platform;
a recess platform abutting said alignment platform;
a rotator molded on top of said alignment platform;
a knob attached atop of said rotator;
a hook connected under said knob bending toward said bottom section and upward; and
an upper platform defining a slot and an opening.

2. A pill bottle for housing a blister card according to claim 1 wherein said top section includes top sidewalls encasing said top panel.

3. A pill bottle for housing a blister card according to claim 2 wherein said bottom section includes bottom sidewalls encasing said bottom panel.

4. A pill bottle for housing a blister card according to claim 3 wherein said rotator includes two curved legs and a curved cross-member wall that allow for flexing and rotational movement.

5. A pill bottle for housing a blister card according to claim 4 wherein a blister card is attached to said pill bottle by vertical posts.

6. A pill bottle for housing a blister card according to claim 5 further comprising at least two alignment snaps located on said top sidewall.

7. A pill bottle for housing a blister card according to claim 6 further comprising at least two alignment snaps located on said bottom sidewall.

8. A child-resistant pill bottle for housing a blister card comprising:
a top section having a top panel and top sidewalls;
a bottom section having a bottom panel and bottom sidewalls;
a center section connected to said bottom section and said top section by hinges;
a child-resistant neck comprising
a top neck having;
an alignment platform connected to said top section;
a rotator located on said alignment platform;
a knob connected to said rotator;
a hook located on said knob;
a bottom neck having;
a recess platform;
an upper platform connected to said recess platform by a wall;
a slot defined within said upper platform; and
an opening defined within said upper platform.

9. A child-resistant pill bottle for housing a blister card according to claim 8 wherein said rotator comprises two curved legs and a curved cross-member so as to provide said rotator with the ability to flex and turn.

10. A child-resistant pill bottle for housing a blister card according to claim 9 further comprising at least two alignment catches located on said top panel and said bottom panel.

11. A child-resistant pill bottle for housing a blister card comprising:
a top section;
a bottom section:
a center section attaching said top section and said bottom section via hinges;
a child-resistant neck having;
a top neck;
a bottom neck;
a partial thread located on said top neck and on said bottom neck that forms a single thread when said top neck and bottom neck are abutted together; and
a cap.

12. The child-resistant pill bottle for housing a blister card according to claim 11 further comprising an alignment tab that is vertically protruding out of said top neck toward said bottom neck.

13. The child-resistant pill bottle for housing a blister card according to claim 14 further comprising a recess tab under said bottom neck.
